(19) United States
(12) Patent Application Publication
(10) Pub. No.: US 2003/0153845 A1
(43) Pub. Date: Aug. 14, 2003

(54) Centering brachytherapy catheter

(76) Inventors: Jeremy Emken, Huntington Beach, CA (US); Russell Hodge, Healdsburg, CA (US)

Correspondence Address: MEDTRONIC AVE, INC., 3576 UNOCAL PLACE, SANTA ROSA, CA, 95403, US

(21) Appl. No.: 10/074,524

(22) Filed: Feb. 11, 2002

(51) Int. Cl. A61B 5/00

(52) U.S. Cl. 600/585

(57) ABSTRACT

An apparatus for radially centering a treatment region of a brachytherapy catheter in a lumen of a body vessel. The centering apparatus includes a monofilament wire-form that is pre-shaped to have an expanded configuration having multiple lobes arranged in a radially symmetrical staggered sequence along the treatment region of the catheter. The centering apparatus also has a collapsed configuration that is formable about the catheter by drawing apart the ends of the wire-form. This abstract is provided to comply with the rules requiring an abstract which will allow a searcher or other reader to quickly ascertain the subject matter of the technical disclosure. It is submitted with the understanding that it will not be used to interpret or limit the scope or meaning of the claims. 37 CFR 1.72(b)

### FIELD OF THE INVENTION

The invention relates to catheters used for localized delivery of therapeutic radiation within a vessel of a patient and, in particular, to a wire-form centering the catheter in the vessel.

### BACKGROUND OF THE INVENTION

Stenosis is a narrowing or constriction of a duct or canal. A variety of disease processes, such as atherosclerotic lesions, immunological reactions, congenital abnormalities and the like, can lead to stenoses of arteries or ducts. In the case of stenosis of a coronary artery, this typically leads to myocardial ischemia. Percutaneous transluminal coronary angioplasty (PTCA), the insertion and inflation of a balloon catheter in a coronary artery to affect its repair, is widely accepted as an option in the treatment of obstructive coronary artery disease. In general, PTCA is used to increase the lumen diameter of a coronary artery that is partially or totally obstructed by a build-up of cholesterol fats or atherosclerotic plaque. In PTCA, a coronary guiding catheter provides a channel from outside the patient to the ostium of a coronary artery. Then, a balloon catheter is advanced over a small diameter, steerable guidewire through the guiding catheter, into the artery, and across the stenosis. The balloon is inflated to expand the narrowing. Dilatation of the occlusion, however, can form flaps, fissures and dissections which threaten abrupt reclosure of the dilated vessel or even perforations in the vessel wall. To treat or prevent such sequelae, tubular stents are often placed within the angioplasty site to scaffold the vessel lumen.

Other invasive vascular therapies include atherectomy (mechanical removal of plaque residing inside an artery), laser ablative therapy and the like. While the stenosis or occlusion is greatly reduced using these therapies, many patients experience a recurrence of the stenosis over a relatively short period. Restenosis, defined angiographically, is the recurrence of a 50% or greater narrowing of a luminal diameter at the site of a prior therapy. Additionally, researchers have found that angioplasty or placement of a stent in the area of the stenosis can irritate the blood vessel and cause rapid reproduction of the cells in the medial layer of the blood vessel, developing restenosis through a mechanism called medial hyperplasia. Restenosis is a major problem which limits the long-term efficacy of invasive coronary disease therapies. Additionally, the rapid onset of restenosis is compounded by the lack of ability to predict which patients, vessels, or lesions will undergo restenosis.

Although the mechanism of restenosis is not fully understood, clinical evidence suggests that restenosis results from a migration and rapid proliferation of a subset of predominately medially derived smooth muscle cells, which is apparently induced by the injury from the invasive therapy. Such injury, for example, is caused by the angioplasty procedure when the balloon catheter is inflated and exerts pressure against the artery wall, resulting in medial tearing. It is known that smooth muscle cells proliferate in response to mechanical stretch and the resulting stimulation by a variety of growth factors. Also, intimal hyperplasia can contribute to restenosis, stimulated by the controlled therapeutic injury. It is believed that such proliferation stops one to two months after the initial invasive therapy procedure but that these cells continue to express an extracellular matrix of collagen, elastin and proteoglycans. Additionally, animal studies have shown that during balloon injury, denudation of endothelial cells can occur, followed by platelet adhesion and aggregation, and the release of platelet-derived growth factor (PDGF) as well as other growth factors. As mentioned above, this mass of tissue can contribute to the re-narrowing of the vascular lumen in patients who have restenosis. It is believed that a variety of biologic factors are involved in restenosis, such as the extent of the tissue injury, platelets, inflammatory cells, growth factors, cytokines, endothelial cells, smooth muscle cells, and extracellular matrix production, to name a few.

It has been found that irradiating the blood vessel walls at the treatment site can reduce or prevent hyperplasia. Accurate control over the amount of radiation is important, since insufficient radiation will not prevent restenosis and excessive radiation can further damage the blood vessel or surrounding tissues. To prevent unnecessary radiation beyond the site of the stenosis, it is preferable to introduce a small radiation source into the treated vessel. There are numerous types of radiation catheters for this purpose.

### BRIEF DESCRIPTION OF THE DRAWINGS

The present invention will be understood and appreciated more fully from the following detailed description taken in conjunction with the appended drawings in which:

FIG. 1 is a plan view of a partial longitudinal cross-section of a catheter according to the invention, with a centering wire expanded against the lumen of a body vessel;

FIG. 2 is an elevation view of the embodiment according to the invention shown in FIG. 1;

FIG. 3 is a transverse sectional view of a catheter according to the invention, taken on line 3-3 of FIG. 1;

FIG. 4 is a transverse sectional view of a catheter according to the invention with an alternative embodiment of the centering wire expanded against the lumen of a body vessel;

FIG. 5 is a transverse sectional view of a catheter according to the invention with another alternative embodiment of the centering wire expanded against the lumen of a body vessel;

FIGS. 6-14 are isometric views of alternative embodiments of the centering wire according to the invention, shown in the expanded configuration;

FIG. 15 is a longitudinal view of the distal portion of a catheter according to the invention, with a centering wire expanded;

FIG. 16 is a longitudinal view of the distal end of the catheter embodiment shown in FIG. 15, with the centering wire collapsed;

FIG. 17 is a longitudinal view of the distal end of an alternative embodiment of a catheter according to the invention, with the centering wire expanded;

FIG. 18 is a longitudinal view of the distal end of the catheter embodiment shown in FIG. 17, with the centering wire collapsed;

FIG. 19 is a longitudinal view of the distal end of another alternative embodiment of a catheter according to the invention, with the centering wire expanded.

### DETAILED DESCRIPTION OF THE INVENTION

An apparatus is provided by the present invention that allows for intraluminal radiation therapy (IRT), also called brachytherapy. Applicant's invention is useful with any type of brachytherapy catheter that delivers a small diameter radiation source to a targeted body vessel. Such catheters may feature a lumen for temporary insertion of a radioactive source such as a fluid, a wire or a pellet, or the catheter may have an electrically activated radiation source built into a treatment region adjacent its distal end. Regardless of the source of radiation, such catheters benefit from a mechanism to center them within the lumen of the body vessel, thus ensuring uniform exposure of the vessel tissue.

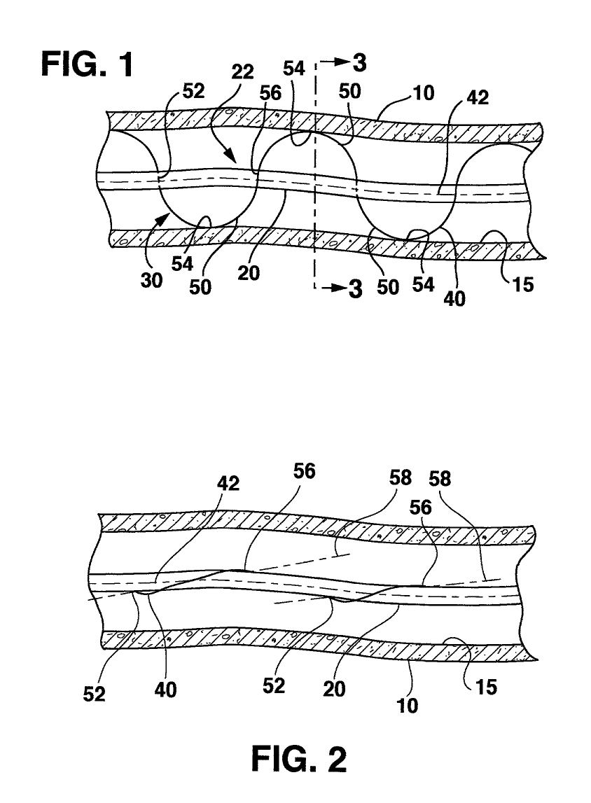

FIGS. 1-3 illustrate a section of body vessel 10 having lumen 15 and brachytherapy catheter 20 extending there through. Catheter 20 may be any type of IRT catheter, such as those described above. Centering wire 30 is mounted about treatment region 22 of catheter 20, and comprises wire-form 40, which is shown in an expanded configuration. The term wire-form is used herein to refer to any pre-formed monofilament that exhibits a shape memory. Other centering wire embodiments, in accordance with the invention, may comprise a conjunction of two or more wire-forms, as will be described below. Examples of materials that are useful for wire-forms in the invention are wires made of nitinol (NiTi) or spring temper stainless steel, or filaments made of thermoplastic polymers. Wire-form 40 comprises a series of lobes 50 arranged along its length. Wire-form 40 also has a center line, which extends axially through the center of catheter 20. To aid in description of the invention, reference will be made to center line 42. According to the invention, the number of lobes 50 in a wire-form can be selected as desired. Variables affecting the design of a particular wire-form can be related to the length of treatment region 22 that needs to be centered, and to the axial length of lobes 50. The axial length of lobes 50 can be defined by the chosen lobe shapes, which may be generally semi-elliptical or semi-circular. Lobes 50 may also be U-shaped, having roughly parallel sides and rounded outer ends. Each lobe 50 has starting segment 52 adjacent one side of center line 42, and ending segment 56, which is axially displaced from starting segment 52 and is adjacent the opposite side of center line 42. Lobe 50 extends radially outwardly from starting segment 52 to apex 54, then continues radially inwardly to ending segment 56. Because lobe 50 starts and ends on opposite sides of center line 42, lobe 50 defines generally flat plane 58, which crosses center line 42 at a slight angle, as shown in FIG. 2. In wire-form 40, lobes 50 are staggered within a single plane such that wire-form 40 may resemble a sine wave with peaks and valleys that are engageable with lumen 15 of body vessel 10.

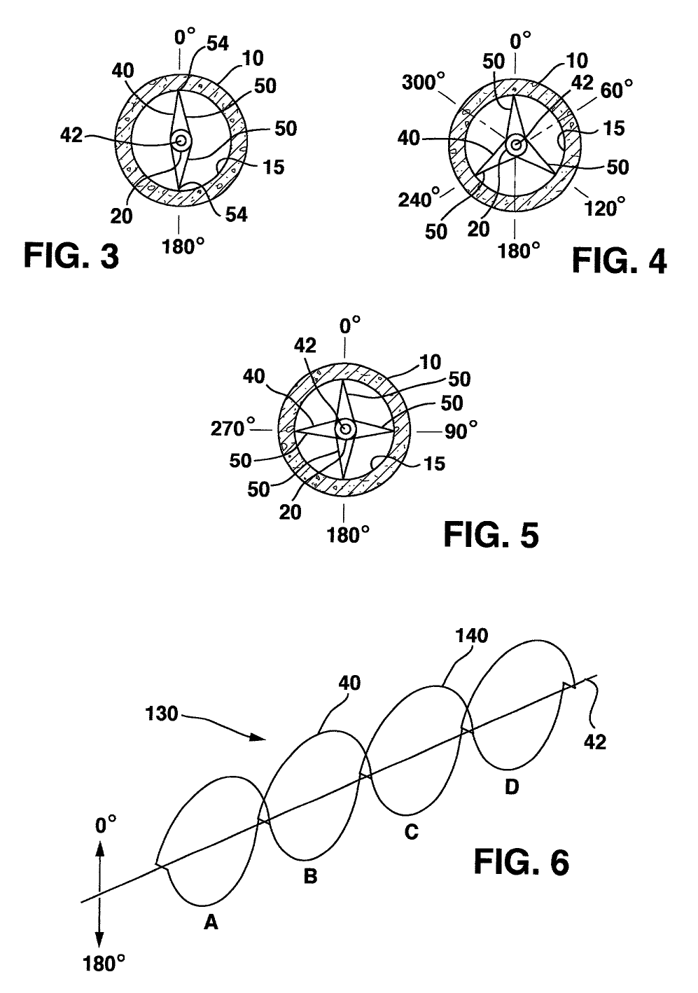

Wire-form 40, and other wire-forms in accordance with the invention, are characterized as having multiple lobes 50 arranged in a radially symmetrical staggered sequence along center line 42. Lobes 50 are termed in a “sequence” because each lobe 50 is formed longitudinally adjacent another lobe 50. The designation “staggered” means that each lobe 50 extends in a different radial direction from adjacent lobe(s) 50. In the case of wire-form 40, which lies generally within a single plane, sequential lobes 50 alternate between 0° and 180° angular positions about center line 42, as shown in FIG. 3. The staggered sequence of lobes 50 is also termed “radially symmetrical” because the angular positions of lobes 50 are equally spaced about center line 42. As discussed above, FIG. 3 shows the radial symmetry of wire-form 40, in which lobes 50 extend from center line 42 in two directions: 0° and 180°. FIGS. 4 and 5 show the radial symmetry of wire-forms in alternative embodiments of the invention, which are discussed in further detail below. In FIG. 4, lobes 50 extend from center line 42 in three equally spaced directions: 0°, 120° and 240°. In FIG. 5, lobes 50 extend from center line 42 in four equally spaced directions: 0°, 90°, 180° and 270°.

Centering wire 30 functions to center catheter 20 in the body vessel as follows. With wire-form 40 in its expanded configuration, apexes 54 contact lumen 15, and the radial symmetry of wire-form 40 tends to retain center line 42 centered in vessel 10. To restrain catheter 20 about center line 42, starting and ending segments 52, 56 prevent their respective contact points with catheter 20 from moving or bending towards lumen 15. For example, as can be seen in FIG. 3, wire-form 40 can effectively prevent catheter 20 from moving laterally, or perpendicular to the plane extending through directions 0° and 180°. Starting and ending segments 52, 56 contact catheter 20 on alternating opposite sides only, such that each contact point can only deter lateral displacement of catheter 20 in one direction. In this example, contact points on the same side are separated by the length of two lobes 50. Alternate embodiments of centering wire 30 are presented below, and feature more contact points and closer separation distances therebetween to achieve better centering performance, especially in curved body vessels. To a lesser extent, wire-form 40 can also act to restrain catheter 20 centered within the plane through directions 0° to 180°, shown in FIG. 3. For catheter 20 to move away from center line 42 towards apex 54 of lobe 50 requires starting and ending segments 54, 56 to twist out-of-plane, or for catheter 20 to bend laterally through lobe 50.

To exemplify the functionality of centering wire 30 and several alternative embodiments, Table 1 shows the angular position of centering lobes 50 for each example. The wire-forms in each of centering wires 30-430 are formed in a single plane. The positions (A-E) refer to lobes 50 of each wire-form 40, in longitudinal sequence. In the first example of Table 1, centering wire 30 comprises wire-form 40 and has been described above.

The second example in Table 1 is centering wire 130, which is shown in FIG. 6 and comprises a conjunction of wire-forms 40, 140. Wire-form 140 is formed as a mirror image of wire-form 40, or it may be considered to have the same shape as wire-form 40 and to be axially displaced by one lobe or by one half-cycle in a sine wave. In centering wire 130, each starting segment 52 of wire-form 40 is directly on the opposite side of catheter 20 from an ending segment 56 of wire-form 140 Thus, each point of contact between catheter 20 and centering wire 130 can deter lateral displacement of catheter 20 in two opposite directions, with contact points on the same side being axially separated only by the length of one lobe 50. Each longitudinal sequence position in centering wire 130 has two lobes 50, arranged directly opposite each other, as indicated in Table 1.

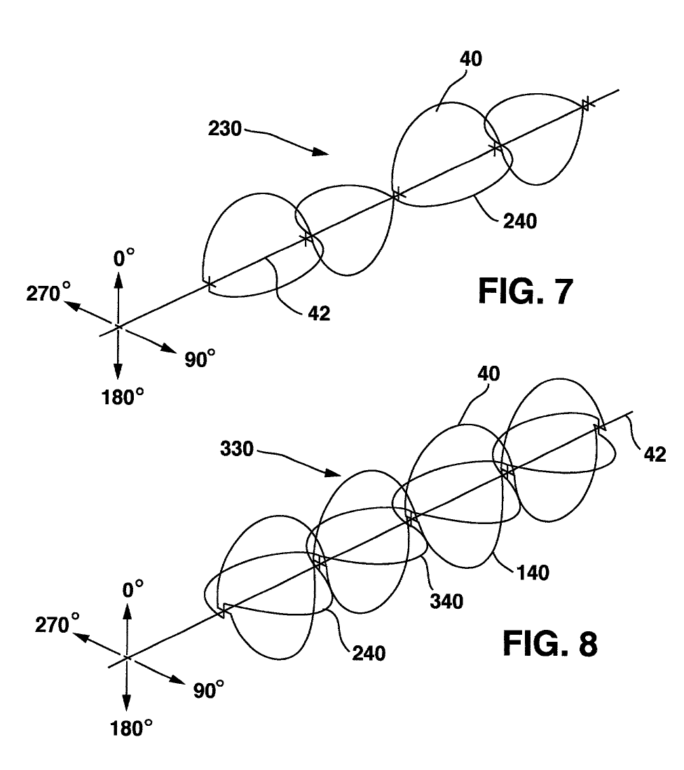

The third example in Table 1 is centering wire 230, which is shown in FIG. 7 and comprises a conjunction of wire-forms 40,240. Wire-form 240 has the same shape as wire-form 40 and is angularly displaced 90° there from. In centering wire 230, each starting segment 52 of wire-form 40 has a starting segment 52 of wire-form 240 displaced 90° there from on catheter 20. Thus, each point of contact between catheter 20 and centering wire 230 can deter lateral displacement of catheter 20 in two orthogonal directions, with contact points on the same side being axially separated by the length of two lobes 50. Each longitudinal sequence position in centering wire 230 has two lobes 50, arranged orthogonally around center line 42, as indicated in Table 1.

TABLE 1
Angular Position (in degrees) of Centering Lobes for
Centering Wires having Wire-Forms in a Single Plane

| Centering | | Longitudinal Sequence Position | | | | |
|---|---|---|---|---|---|---|
| Wire | Wire-Form | A | B | C | D | E |
| 30 | 40 | 0 | 180 | 0 | 180 | 0 |
| 130 | 40 | 0 | 180 | 0 | 180 | 0 |
| | 140 | 180 | 0 | 180 | 0 | 180 |
| 230 | 40 | 0 | 180 | 0 | 180 | 0 |
| | 240 | 90 | 270 | 90 | 270 | 90 |
| 330 | 40 | 0 | 180 | 0 | 180 | 0 |
| | 140 | 180 | 0 | 180 | 0 | 180 |
| | 240 | 90 | 270 | 90 | 270 | 90 |
| | 340 | 270 | 90 | 270 | 90 | 270 |
| 430 | 40 | 0 | 180 | 0 | 180 | 0 |
| | 440 | 120 | 300 | 120 | 300 | 120 |
| | 540 | 240 | 60 | 240 60 | 240 | |

The fourth example in Table 1 is centering wire 330, which is shown in FIG. 8 and comprises a conjunction of wire-forms 40, 140, 240, 340. In centering wire 330, starting segments 52 of wire-forms 40, 140, 240, 340 are equally spaced around catheter shaft 20. Thus, each point of contact between catheter 20 and centering wire 330 can deter lateral displacement of catheter 20 in four directions, with contact points on the same side being axially separated by only the length of one lobe 50. Each longitudinal sequence position in centering wire 330 has four lobes 50,which are equally spaced around center line 42, as indicated in Table 1. With its four lobes 50 and four adjacent contact points, centering wire 330 provides a high degree of centering, and constructing this embodiment requires four separate wire-forms 40, 140, 240, 340.

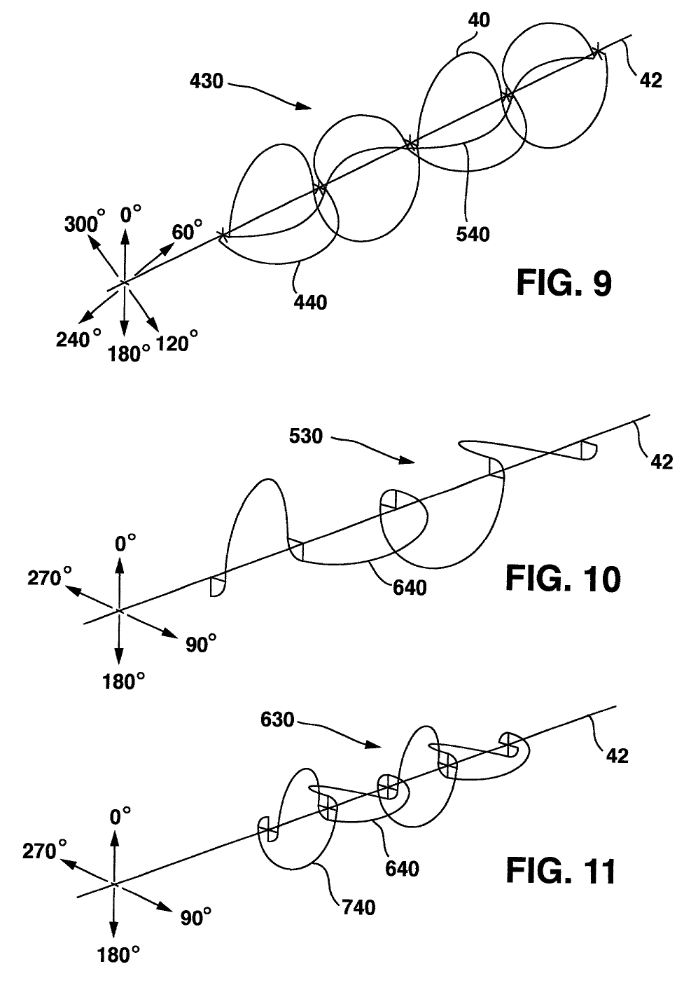

The last example in Table 1 is centering wire 430, which is shown in FIG. 9 and comprises a conjunction of wire-forms 40, 440, 540. Wire-forms 440, 540 have the same shapes as wire-form 40 and are angularly displaced 120° and 240° there from, respectively. In centering wire 430, starting segments 52 of wire-forms 40, 440, 540 are equally spaced around catheter shaft 20. Thus, each point of contact between catheter 20 and centering wire 430 can deter lateral displacement of catheter 20 in three directions, with contact points on the same side being axially separated by the length of two lobes 50. Each longitudinal sequence position in centering wire 430 has three lobes 50,which are equally spaced around center line 42, as indicated in Table 1. In each sequence position, the lobes 50 are angularly displaced 60° relative the adjacent position.

In all of the examples in Table 1, the wire-forms are formed in a single plane. In other alternative embodiments of centering wires, the wire-forms can be formed in multiple planes. In such cases, starting and ending segments 52, 56 are wrapped at least partially around center line 42, and ending segment 56 of one lobe 50 may also be starting segment 52 of an adjacent lobe 50. Table 2 shows the angular position of centering lobes 50 for alternative embodiment centering wires 530-930, in which all of the wire-forms would be formed in multiple planes.

The first example in Table 2 is centering wire 530, which is shown in FIG. 10. Centering wire 530 comprises wire-form 640, in which axially adjacent lobes 50 are arranged 90° apart. Although lobes 50 are arranged in four staggered, equally-spaced directions, the partial wrapping of starting and ending segments 52, 56 around catheter 20 provides a quarter-circle range of centering support at each contact point, as compared to the unidirectional support offered by a single-plane wire-form such as wire-form 40, discussed above.

The second example in Table 2 is centering wire 630, shown in FIG. 11. Centering wire 630 comprises a conjunction of wire-forms 640, 740. Wire-form 740 is similar to wire-form 640, but it is axially displaced with respect thereto by two lobe positions. As a result, each longitudinal lobe position along centering wire 630 can support a catheter in two opposite directions, either 0° and 180° or 90° and 270°.

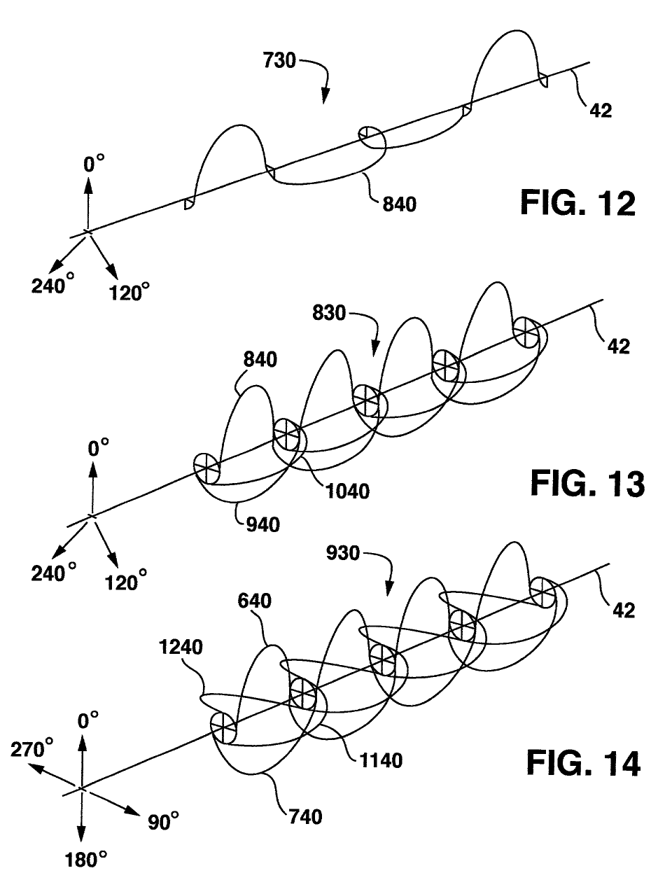

The third example in Table 2 is centering wire 730, shown in FIG. 12. Centering wire 730 comprises wire-form 840, in which axially adjacent lobes 50 are arranged 120° apart. In wire-form 840, lobes 50 are arranged in three staggered, equally-spaced directions, and the partial wrapping of starting and ending segments 52, 56 around catheter 20 provides a one third of a circle range of centering support at each contact point with a catheter.

The fourth example in Table 2 is centering wire 830, shown in FIG. 13. Centering wire 830 comprises a conjunction of wire-forms 840, 940, 1040. Wire-forms 940 and 1040 are similar to wire-form 840, but they are axially displaced by one and two lobe positions, respectively. As a result, each longitudinal lobe position along centering wire 830 can support a catheter in three equally spaced directions.

The fifth example in Table 2 is centering wire 930, shown in FIG. 14. Centering wire 930 comprises a conjunction of wire-forms 640, 740, 1140, 1240. Wire-forms 1140, 1240 are similar to wire-form 640 but they are axially displaced with respect thereto by one lobe position in each axial direction. As a result, each longitudinal lobe position along centering wire 630 can support a catheter in four opposite directions, 0°, 180°, 90° and 270°.

TABLE 2
Angular Position (in degrees) of Centering Lobes for
Centering Wires Having Wire-Forms in Multiple Planes

| | | Longitudinal Sequence Position | | | | |
|---|---|---|---|---|---|---|
| Example | Wire-Form | A | B | C | D | E |
| 530 | 640 | 0 | 90 | 180 | 270 | 0 |
| 630 | 640 | 0 | 90 | 180 | 270 | 0 |
| | 740 | 180 | 270 | 0 | 90 | 180 |
| 730 | 840 | 0 | 120 | 240 | 0 | 120 |
| 830 | 840 | 0 | 120 | 240 | 0 | 120 |
| | 940 | 120 | 240 | 0 | 120 | 240 |
| | 1040 | 240 | 0 | 120 | 240 | 0 |
| 930 | 640 | 0 | 90 | 180 | 270 | 0 |
| | 1140 | 90 | 180 | 270 | 0 | 90 |
| | 740 | 180 | 270 | 0 | 90 | 180 |
| | 1240 | 270 | 0 | 90 | 180 | 270 |

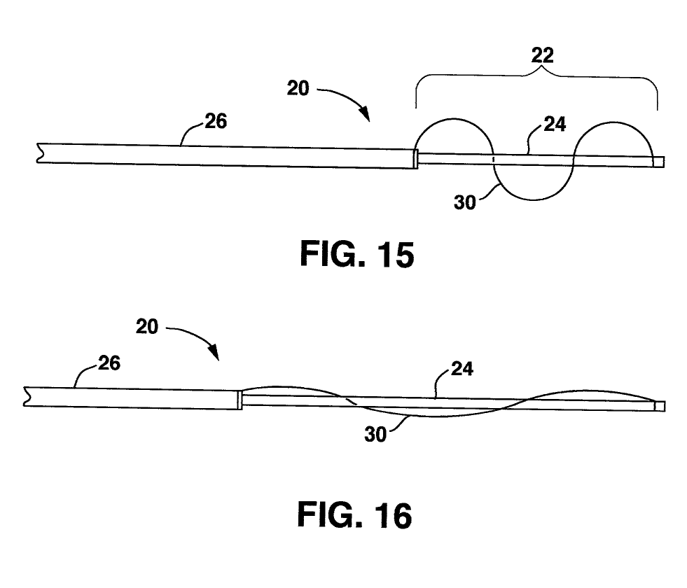

FIG. 15 shows catheter 20 and centering wire 30, which is in an expanded configuration. Catheter 20 includes distal treatment region 22, which is adapted to irradiate a body vessel, as described above. Catheter 20 may be fabricated from typical components such as extruded and/or braided tubes, and may also include tubing or stiffening wires made of metal(s). Specific materials for such components are common knowledge to those of ordinary skill in the field of catheters. Catheter 20 also has shaft 24 and actuator sleeve 26, which slidably surrounds a proximal portion of shaft 24. Centering wire 30 has its distal end coupled to the distal end of shaft 24, and its proximal end is coupled to the distal end of actuator sleeve 26. Coupling(s) between centering wires and catheter elements may comprise any suitable means, such as adhesives or the sandwiching of wire portions between layers of melt-bonded thermoplastic polymers.

FIG. 16 shows catheter 20 and centering wire 30 of FIG. 15 in a collapsed configuration, achieved by drawing actuator sleeve 26 proximally along shaft 24 to draw or stretch apart the proximal and distal ends of centering wire 30. The collapsed configuration features a low profile suitable for insertion, withdrawal or repositioning of the apparatus within body vessels of a patient. In the collapsed configuration, centering wire 30 may be generally wrapped around shaft 24 because lobes 50 start and end on opposite sides of center line 42. Transformation between the collapsed and expanded configurations is repeatably reversible, as often as necessary during treatment of the patient. Shape memory of the expanded configuration is set into wire-form 40, typically by winding the monofilament in a fixture and heating the assembly under conditions of time and temperature suitable for the chosen material.

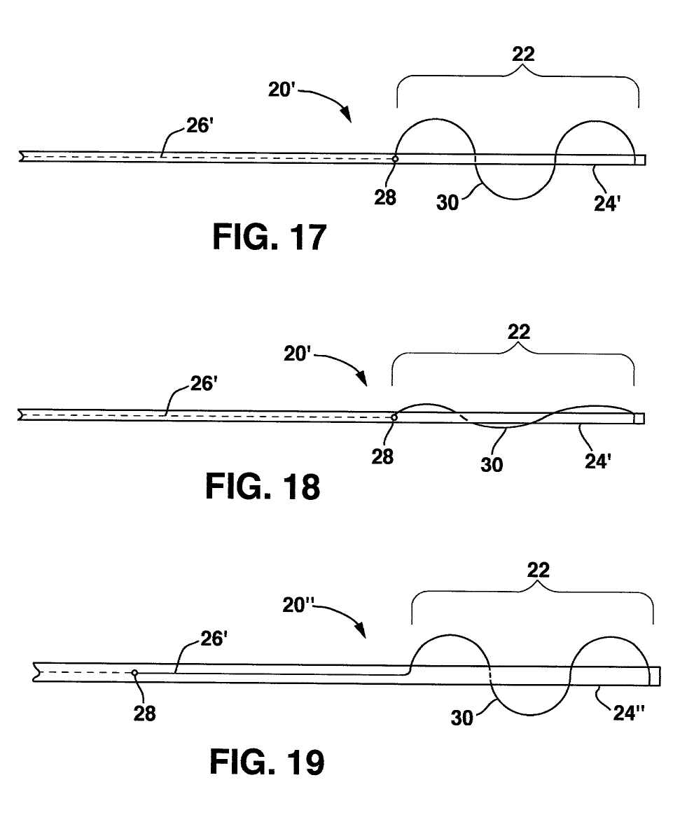

FIG. 17 shows an alternative embodiment of the invention having catheter 20′ and centering wire 30. Catheter 20′ includes distal treatment region 22 and shaft 24′. Port 28 is an opening through the wall of shaft 24′ and is located adjacent the proximal end of distal treatment region 22. The distal end of centering wire 30 is coupled to the distal end of shaft 24′. The proximal end of centering wire 30 is coupled to the distal end of actuator filament 26′, which slidably extends through a lumen (not shown) in shaft 24′.

FIG. 18 shows catheter 20′ and centering wire 30 of FIG. 17 in a collapsed configuration, achieved by pulling actuator filament 26′ proximally through shaft 24′ to draw or stretch apart the proximal and distal ends of centering wire 30. As it is transformed from the expanded configuration to the collapsed configuration, a proximal portion of centering wire 30 is drawn through port 28 into the lumen of shaft 24′.

FIG. 19 shows another alternative embodiment of the invention having catheter 20″ and centering wire 30. In catheter 20″, port 28 is located proximally of distal treatment region 22 such that, when centering wire 30 is transformed from its expanded configuration to its collapsed configuration, none of its pre-formed lobes needs to be drawn through port 28. In catheters 20′, 20″ centering wire 30 and actuator filament 26′ may be separate elements that are coupled together, in which case, the coupling between centering wire 30 and actuator filament 26′ may always lie inside of shaft 24′, or it may always lie outside of shaft 24′, or the coupling may pass through port 28 during transformation of centering wire 30 between expanded and collapsed configurations. Alternatively, centering wire 30 and actuator filament 26′ may be made as separate portions of a single component. Catheters 20, 20′ may both feature a locking mechanism (not shown) at the proximal end, which can maintain the relative positions of shaft 24 and actuator sleeve 26, or shaft 24′ and actuator filament 26′. Such a locking mechanism can temporarily hold the apparatus in either the expanded or collapsed configurations.

While catheters 20, 20′, and 20″ are each shown, for simplicity, with centering wires that have only single wire-forms, it is understood from the examples above that two or more wire-forms may be mounted about the distal treatment region of the brachytherapy catheter. In such multi wire-form embodiments, the wire-forms may be mounted to the catheter independently of each other, or they may be joined at one or both ends, which may simplify the attachment between the centering wire and an actuator. As discussed above, multiple wire-form embodiments can provide radial centering in more than one direction, which may be advantageous in treating tortuous body vessels.

While the invention has been particularly shown and described with reference to the several disclosed embodiments thereof, it will be understood by those skilled in the art that various changes in form and detail may be made therein without departing from the spirit and scope of the invention. For example, catheters 20, 20′, and 20″ may be of several types, including over-the-wire (OTW), single operator or rapidly exchangeable (RX), and fixed-wire (FW).

We claim:

1. An apparatus for radially centering a treatment region of a brachytherapy catheter in a lumen of a body vessel, the catheter having a center line, the apparatus comprising a monofilament wire-form having:
i) proximal and distal ends,
ii) an expanded configuration preformed with multiple lobes arranged in a radially symmetrical staggered sequence along the center line, each lobe extending from the center line to an apex engageable with the lumen of the body vessel, and
iii) a collapsed configuration formable compactly about the center line by drawing apart the wire-form proximal and distal ends.

2. The apparatus of claim 1 wherein the wire-form, when in the expanded configuration, has a length sufficient to extend over the treatment region of the brachytherapy catheter.

3. The apparatus of claim 2 wherein the wire-form proximal and distal ends are attachable to the brachytherapy catheter.

4. The apparatus of claim 1 wherein the expanded configuration of the wire-form comprises a distal portion of the wire-form, the distal portion having a length sufficient to extend over the treatment region of the catheter, the wire-form further comprising a proximal portion extending from the distal portion at least to a proximal end of the catheter, the proximal portion being capable of drawing the wire-form proximal end away from the wire-form distal end.

5. The apparatus of claim 1 wherein the lobes are generally disposed in one plane extending through the center line.

6. The apparatus of claim 5 wherein the wire-form is adapted for conjunction about the catheter with at least one additional wire-form adapted for radially centering a treatment region of a catheter in a lumen, such that a combination of the wire-forms has a radially symmetrical sequence of lobes arranged along the center line.

7. The apparatus of claim 6 wherein the radially symmetrical sequence of lobes is staggered along the center line.

8. The apparatus of claim 1 wherein the lobes are generally disposed in at least two radial directions extending through the center line.

9. The apparatus of claim 1 wherein the lobes have shapes that are generally semi-circular or semi-elliptical.

10. The apparatus of claim 1 wherein each lobe has starting and ending segments that are at least partially wrapped around the center line

11. The apparatus of claim 10 wherein the ending segment of one lobe is also the starting segment of an adjacent lobe.

12. The apparatus of claim 10 wherein the starting and ending segments of each lobe are on opposite sides of the center line.

13. A catheter for brachytherapy treatment of a body vessel from within a lumen thereof, the catheter comprising:
an elongate flexible shaft having a distal end and a radiation source located within a distal treatment region;
an actuator element having a distal end and being slidably disposed along the shaft;
a monofilament wire-form mounted about the distal treatment region for radially centering the treatment region within the lumen, the wire-form having:
i) a distal end coupled to the shaft adjacent the distal end thereof,
ii) a proximal end coupled to the distal end of the actuator,
iii) an expanded configuration preformed with multiple lobes arranged in a radially symmetrical staggered sequence along the distal treatment region, each lobe extending from adjacent the distal treatment region to an apex engageable with the lumen of the body vessel, and
iv) a collapsed configuration formable compactly about the shaft by sliding the actuator proximally with respect to the shaft to draw apart the wire-form proximal and distal ends.

14. The catheter of claim 13 wherein the lobes are generally disposed in one plane extending through the distal treatment region.

15. The catheter of claim 13 further comprising at least one additional wire-form adapted for radially centering a treatment region of a catheter in a lumen, such that a conjunction of the wire-forms has a radially symmetrical sequence of lobes arranged along the distal treatment region.

16. The apparatus of claim 15 wherein the radially symmetrical sequence of lobes is staggered along the distal treatment region.

17. The catheter of claim 13 wherein the lobes are generally disposed in at least two radial directions extending through the distal treatment region.

18. The catheter of claim 13 wherein the lobes have shapes that are generally semi-circular or semi-elliptical.

19. The catheter of claim 13 wherein each lobe has starting and ending segments that are at least partially wrapped around the distal treatment region.

20. The catheter of claim 19 wherein the starting segment of one lobe is also the ending segment of an adjacent lobe.

21. The catheter of claim 19 wherein the starting and ending segments of each lobe are on opposite sides of the distal treatment region.

22. The catheter of claim 13 wherein the actuator is a tubular sleeve disposed about the shaft.

23. The catheter of claim 13 wherein the actuator is a filament at least partially disposed within the shaft.
